(19) United States
(12) Patent Application Publication
(10) Pub. No.: US 2009/0311742 A1
(43) Pub. Date: Dec. 17, 2009

(54) Phage-Derived Vectors and Methods for Protein Expression

(76) Inventor: David Tabaczynski, Groton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/884,591

(22) PCT Filed: Feb. 17, 2006
(86) PCT No.: PCT/US06/05630

§ 371 Date: Aug. 17, 2007

Related U.S. Application Data
(60) Provisional application No. 60/655,218, filed on Feb. 22, 2005.

(51) Int. Cl. C12P 21/06; C12N 7/00; C12N 15/74; C12N 1/21

(52) U.S. Cl. 435/69.1; 435/235.1; 435/475; 435/252.3

(57) ABSTRACT

A novel system, including vectors, phage particles, host cells and methods of use, for expressing one or more polypeptides of interest in prokaryotes is described.

### RELATED APPLICATION

This application claims the benefit of U.S. Provisional Application No. 60/655,218, filed on Feb. 22, 2005. The entire teachings of this application are incorporated herein by reference.

### BACKGROUND OF THE INVENTION

Certain protein expression systems have been used in microorganisms. These systems are based on recombinant microorganisms and genetically engineered vectors, which are typically used in combination to improve protein yields. The vector typically contains components that allow it to maintain a foreign (exogenous) gene in the host cell of choice and to control expression of the associated heterologous protein.

Plasmid vectors are most commonly used in methods of gene expression vector while viral vectors have yet to see widespread adoption outside the field of cloning. Despite its market dominance, plasmid-based systems have many problems related to expression control, plasmid stability, and expression yield that have led to the invention of many additional vector components. These vector components, together with associated recombinant microorganisms, make up the majority of common methods in current use for recombinant protein expression.

Methods for protein expression suffer from many problems including, for example, poor yields, host cell toxicity, and plasmid loss. Aside from poor yields due to inefficient expression, aggregation, and slow host cell growth, heterologous proteins in many cases, when expressed, are toxic to host cells. Inducible expression systems have been devised to control the timing of expression to reduce toxicity, however, despite many novel transcriptional control systems, uninduced expression from leaky promoters can be very problematic. In the case where a gene product is toxic or regulatory in nature, the transformed host cell may grow poorly, have plasmid instability, or low levels of exogenous gene expression.

To improve plasmid stability, various methods of selection are used to ensure the plasmid is stabilized in the host cell during culture growth and protein expression. The most common vector component used to ensure plasmid stability is antibiotic resistance. Alternatively, host cell strains can be developed that harbor mutations to essential genes. Plasmids carrying the required gene in trans can complement the mutated host such that a loss of the plasmid would result in a loss of cell viability. Alternately, the heterologous gene can be placed within the host genome at multiple copies to ensure the gene will not be lost during culture growth. For such plasmid-based vector expression systems, the number of copies of the vector can be directly related to expression levels in bacteria as well as yeast.

Expression in bacteria by these methods faces the problem that, even if high levels of gene transcription and eventual translation can be achieved, E. coli-based systems can only produce moderate amounts of protein before the proteins begin to aggregate in the cytoplasm and form insoluble inclusion bodies. Purification of inclusion bodies can be difficult at many steps of the process-extraction, solubilization of inclusion bodies, and refolding to an active form. Extraction is complicated by the need to break up the host cells to release the soluble protein and the insoluble inclusion bodies. Solubilization is complicated due to the use of organic solvents that need to be removed at future purification steps and results in significant loss of overall yield. Protein refolding and renaturation can alleviate this problem to some extent, however, expensive and toxic agents are required (for a discussion of solubility problems, see, for example, Villaverde, A. and Carrio, M., 2003, Biotechnol. Lett., 25:1385-95; Rudolph, R. and Lilie, H., 1996, FASEB J., 10:49-56; Misawa, S. and Kumagai, I., 1999, Biopolymers, 51:297-307; Schmidt, F., 2004, Appl. Miciobiol. Biotechnol., 65:363-72; Stader, J. and Silhavy, T., 1990, Meth. Enzymol., 185:166-187; Butt, T. et al., 2003, U.S. Patent Application No. 20030153045).

An alternative to plasmid-based expression systems are viral- or phage-based system;, the bulk of phage vectors have been designed from bacteriophage lambda (λ) (Christensen. A., 2001, Mol. Biotechnol., 17:219-24; Chauthaiwale, V. et al., 1992, Microbiol. Rev., 56:577-91). Phage λ is temperate and has the ability to take a lysogenic or lytic pathway after it has infected its host cell. In addition, many of the genes of phage λ can be removed and still allow for the lysogenic or lytic pathways of development to occur in the host cell after infection (Echols, H., 1972, Annu. Rev. Genet., 6:157-90; Frischauf, A. et al., 1983, J. Mol. Biol., 170:827-42). This “extra space” in the recombinant phage can be used to carry genes of interest.

In terms of the issues related to gene stability, phage-based viral vectors can be used in two ways, as a lysogenic vector or as a lytic vector. The lysogenic method utilizes recombination sites to incorporate the phage genome into the host cell genome. In this method any gene that is placed in the phage genome will be inserted into the host cell genome and replicated during host cell division in culture. For λ-based expression vectors, constitutive expression of the CI protein represses lytic development and maintains the lysogenic state of the prophage. An example of a phage lysogen used for gene delivery is in the creation of novel host strains such as λ prophage used to carry the T7 RNA polymerase for use in the pET vector system (Studier, F. and Moffat, B., 1986, J. Mol. Biol., 189:113-30; Studier et al., 1990, U.S. Pat. No. 4,952,496; Studier et al., 1997, U.S. Pat. No. 5,693,489; Studier et al., 1999, U.S. Pat. No. 5,869,320). However, the prophage (lysogen) method of gene delivery differs little from the plasmid vector since it does not solve the issues related to leaky promoters. Therefore a fully lytic vector that transiently infects a host cell would be a better vector for expression of toxic genes.

Attempts to create truly lytic phage-based vector that delivers a gene of interest into the cell have been tested, but yields have been lower than plasmid-based systems (Moir, A. and Brammar, W., 1976, Mol. Gen. Genom., 149:87-99; Mory et al., 1984, G.B. Patent No. 2,130,222A). Because of low yields, phage vectors have been more commonly used to infect cell lines to induce expression of genes located on plasmids as a means to obtain high levels of transient expression. In particular, the use of a phage infection in combination with a host cell that has been transformed with a plasmid vector has been tried as a means to enhance expression levels.

Phage-based vectors can overcome many of the problems associated with plasmid vectors by their very nature. Most notably, viral vectors have been used to overcome the issues surrounding transcriptional control, promoter leakiness, and ultimately gene stability through the use of transient gene delivery and expression. However, the low yield exhibited by phage-based viral vectors indicates there is a need for a high yield, preferably lytic phage-based expression system.

### SUMMARY OF THE INVENTION

This invention relates to a new and improved expression vector system capable of expressing exogenous genes and producing proteins in bacterial hosts, such as, for example, E. coli. The vectors and methods described herein allow transient gene delivery and protein expression using a phage-based vector. The phage-based viral vectors described herein can been used to enhance protein expression yield by, for example, precisely controlling the timing of phage genome packaging, phage particle production and host cell lysis. The vectors described herein can switch from lytic growth and expression without lysis, to host cell lysis through the use of a conditional (inducible) promoter to control transcription and expression of the “late” phage genes. Accurate control of lysis allows one of skill in the art, for example, to maximize expression prior to lysis and subsequent release of the expressed protein into the bacterial culture medium.

In one embodiment of the present invention, the invention is directed to a phage-derived vector for cloning or expressing a heterologous polypeptide of interest in a bacterial cell growing in culture, said vector comprising one or more factors that direct expression of a polypeptide of interest and at least one conditional promoter operably linked to coding sequences expressing one or more late phage transcripts, wherein expression of the one or more late phage transcripts cause host cell lysis, thereby releasing the expressed polypeptide of interest into the bacterial culture.

In one embodiment, the present invention is directed to a phage particle comprising a phage-derived vector for cloning or expressing a heterologous polypeptide of interest in a bacterial cell growing in culture, said vector comprising one or more factors that direct expression of a polypeptide of interest and at least one conditional promoter operably linked to coding sequences expressing one or more late phage transcripts, wherein expression of the one or more late phage transcripts cause host cell lysis, thereby releasing the expressed polypeptide of interest into the bacterial culture.

In one embodiment, at least one conditional promoter is responsive to a chemical signal or a temperature signal, thereby inducing expression from the conditional promoter. In one embodiment, the vector further comprises one or more operator sequences that bind a repressor protein that is affected by a chemical signal, e.g., LacI. In one embodiment, the vector further directs expression of one or more factors that cause host RNA polymerase to selectively start transcription from pre-selected promoters, e.g., wherein one or more factors is selected from the group consisting of: AsiA, MotA, sigma factors, and anti-sigma factors. In one embodiment, the host RNA polymerase initiates transcription at a vector promoter, for example, wherein the vector promoter is operably linked to a sequence encoding the polypeptide of interest. In one embodiment, the vector further comprises one or more promoters that are recognized by an exogenous RNA polymerase, for example, the bacteriophage T7 promoter. In another embodiment, the sequence that encodes the polypeptide of interest is under the control of a promoter that is recognized by the exogenous RNA polymerase.

In another embodiment, the invention is directed to a method for expressing a polypeptide of interest, comprising: (a) infecting a host bacterial culture with a phage particle comprising a phage-derived vector, said vector comprising one or more factors that direct expression of a polypeptide of interest and at least one conditional promoter operably linked to coding sequences expressing one or more late phage transcripts, wherein expression of the one or more late phage transcripts cause host cell lysis; (b) growing the infected bacteria under conditions such that a polypeptide of interest is expressed; and (c) inducing lysis of the cells by inducing expression of the late phage genes, thereby releasing the expressed polypeptide of interest into the culture medium. In one embodiment, induction in (c) is by a chemical signal or a change in temperature. In another embodiment, the vector further comprises one or more operator sequences that bind a repressor protein that is affected by a chemical signal, e.g., LacI. In one embodiment, the vector further directs expression of factors that cause host RNA polymerase to selectively start transcription from a pre-selected promoter, for example, wherein one or more factors is selected from the group consisting of: AsiA, MotA, sigma factors, and anti-sigma factors. In one embodiment, the host RNA polymerase initiates transcription at a vector promoter, for example, wherein the vector promoter is operably linked to a sequence encoding the polypeptide of interest. In one embodiment, the vector further comprises one or more promoters that are recognized by an exogenous RNA polymerase, e.g., the bacteriophage T7 promoter. In another embodiment, one or more genes of interest are expressed in trans with respect to the vector, for example, wherein the one or more genes of interest are expressed from a source selected from the group consisting of: an endogenous gene of interest, a gene of interest inserted into the host genome from a lysogenic vector or through recombination, a gene of interest placed in a plasmid vector, and a gene of interest placed in a viral vector. In another embodiment, the host cell comprises a gene that encodes an exogenous RNA polymerase, e.g., wherein the RNA polymerase is from bacteriophage T7. In another embodiment, one or more control elements are expressed in the host cell, for example, wherein expression of the one or more control elements is from a source selected from the group consisting of: an endogenous gene of interest, a gene of interest inserted into the host genome from a lysogenic vector or through recombination, a gene of interest placed in a plasmid vector, and a gene of interest placed in a viral vector. In one embodiment, the one or more control elements is selected from the group consisting of: AsiA, MotA, sigma factors, anti-sigma factors, transcriptional repressors, altered transcriptional repressors, transcriptional activators and RNA polymerase phosphorylases.

### BRIEF DESCRIPTION OF THE DRAWINGS

FIG. 1 is a drawing depicting the genomic map of the wild-type lambda phage based on the reference sequence NC_{—}001416. This drawing is annotated with key genes and regions as described in this invention.

FIG. 2 is a drawing depicting the genomic map the recombinant lambda phage as it has been altered to carry out the functions represented in this invention. The expression and control cassette placement are shown (see Example 1).

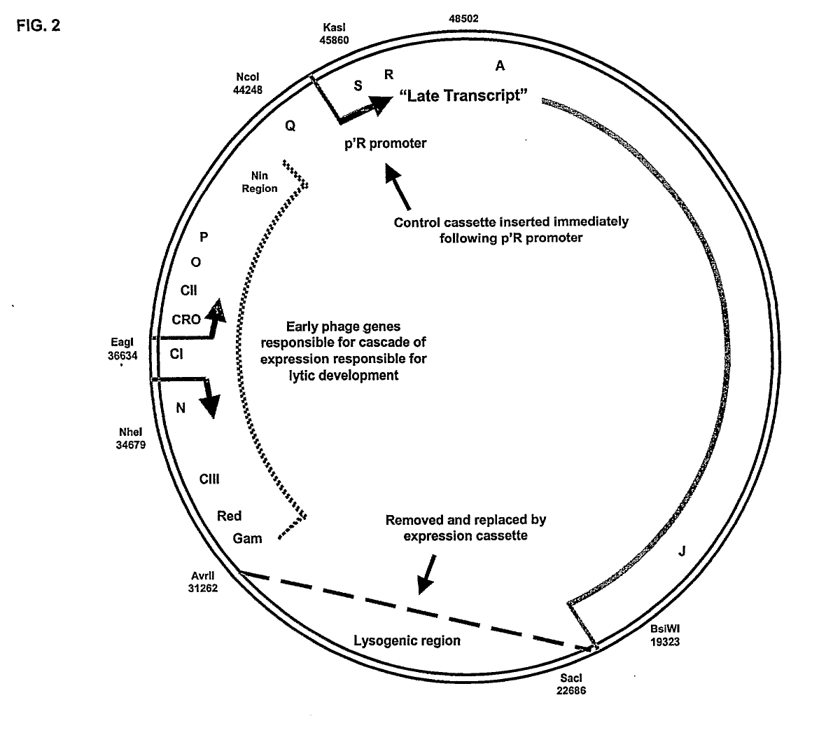

FIG. 3 is a drawing detailing how any lambda-lice temperate phage could be rearranged to make an expression system. Specifically, the method in which the control cassette is used to control expression of late phage gene transcription is shown.

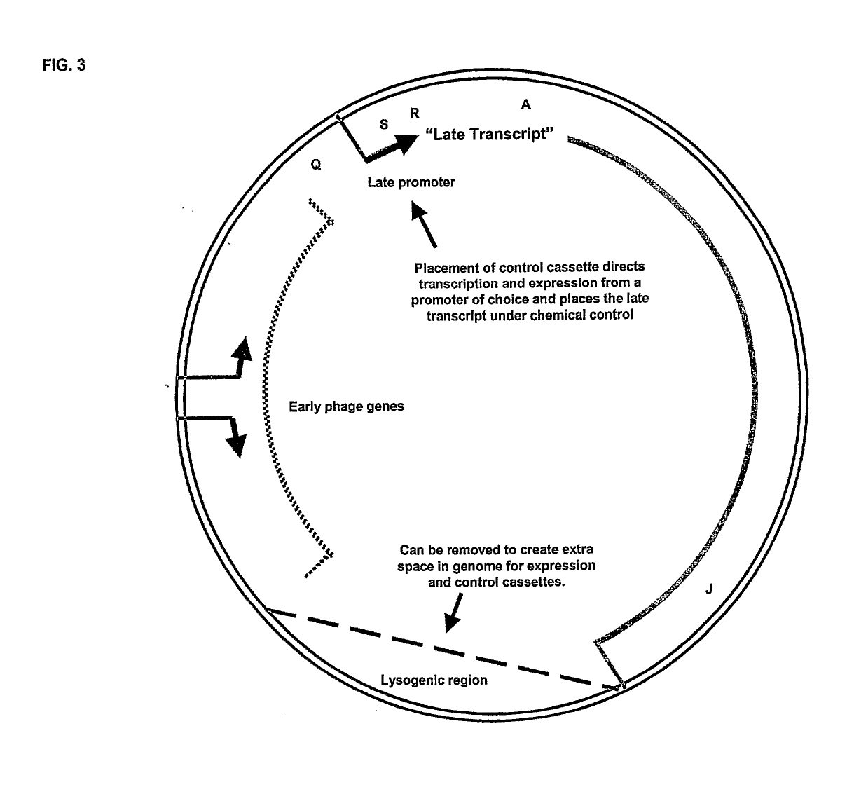

FIG. 4 is a diagram of the control cassette containing intrinsic late promoter, control proteins, and novel late promoter that is under direction of control proteins and repressed by operator sequences. AsiA and MotA genes are placed under control of the lambda p′R promoter. LacI biding operators to repress the T4 middle promoter used to design the novel late promoter.

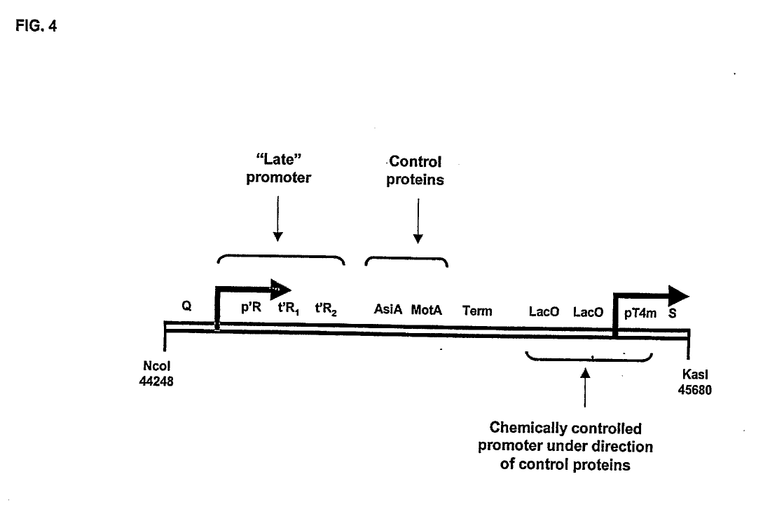

FIG. 5 is a diagram of the expression cassette containing a T4 middle promoter, a multiple cloning site (MCS) and a reporter gene. A gene of interest or other control elements or linkers could be incorporated into the expression cassette using the MCS.

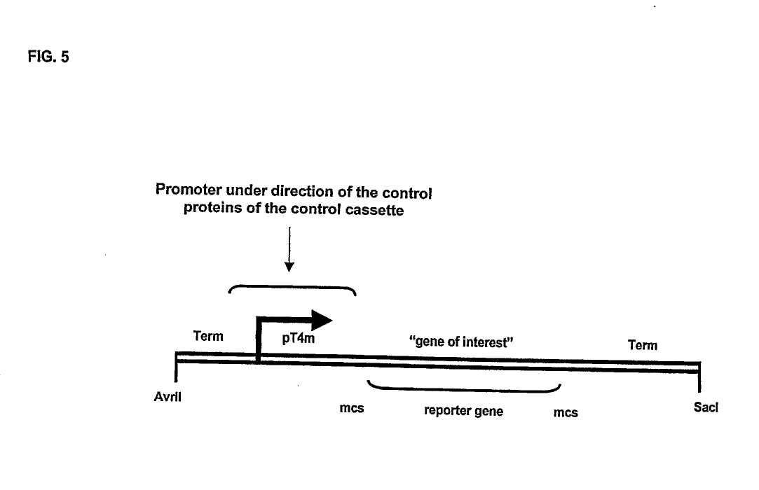

### DETAILED DESCRIPTION OF THE INVENTION

The vectors described herein are recombinant vectors that allow for methods of protein expression in prokaryotes involving infecting host cells with a lytic phage-based viral vector under conditions such that the late viral transcripts necessary for the production of viral capsid and host lysis proteins are repressed. High expression levels of a protein of interest is directed from strong promoters while late viral and host gene transcription is repressed using a conditional (inducible) promoter. As used herein, “gene” refers to at least a coding sequence that codes for a unique polypeptide, and, optionally, genetic regulatory elements necessary for expression. As used herein, “expression” refers to products of transcription or translation, e.g., mRNA or polypeptides. Expression of more than one product can be in “cis”, e.g., where all expression products are expressed from the same molecule, or in “trans”, e.g., where at least one expression product is expressed from a gene located on a separate DNA molecule. Induction of the late transcripts, e.g., by chemical or temperature induction, results host cell lysis and release of expressed protein into the culture medium, as well as restoration of competence to the recombinant phage vector and subsequent production of phage progeny. The precise control of the late vector transcripts required for expression of the capsid, packaging, and lysis proteins that are necessary for packaging and release of new phage progeny allow for high yields of even toxic heterologous polypeptides of interest.

Inducible promoters are known in the art that allow for the precise control of timing of gene expression. For example, chemically-inducible promoters, e.g., inducible by, for example, IPTG, and temperature-inducible promoters, e.g., heat shock promoters, are used for inducible expression. Placing late vector genes under the control of an inducible promoter allows for the précis induction of host cell lysis and viral packaging.

Improved expression yields using such a vector can be achieved using methods known in the art, as well as methods unique to these vectors. For example, the vectors described herein allow one of skill in the art to grow host cell cultures to maximum density and/or cell size prior to infection. Upon infection, infected cells can be maintained without lysis, while heterologous expression occurs. After maximum expression is achieved, lysis can be induced, thereby allowing for a high yield of the expressed heterologous protein into the host cell medium, where it is easily recovered. One advantage of such a technique is that host cells have already achieved maximum density prior to infection, so even if the expression of heterologous protein is toxic to the host cell, high yields are still achieved.

The vectors described herein can direct expression of, for example, a gene of interest or a regulatory factor that is expressed in trans. For example, a gene of interest can be inserted into the host genome or carried on a separate plasmid or viral vector, and is only transcribed upon signals received from the vector of the invention. The phage-derived vector “directs” expression by, for example, providing signals to initiate expression of the genes in trans. The phage vector can control release of the expressed product into the culture medium by, for example providing signals to initiate late phage genes expression or host cell lysis in trans.

The vectors described herein can also utilize elements and methods known in the art for increasing heterologous expression from phage-based viral vectors. For example, elements can be incorporated into the vector that help to release soluble protein into the culture and to suppress host cell function while maintaining expression from exogenous genes located on the phage or associated plasmid. In addition, genes can be incorporated into the vectors described herein that direct RNA polymerase to solely transcribe the desired gene of interest, e.g., through the use of sigma or anti-sigma factors, or through the use of exogenous RNA polymerase and any associated host RNA polymerase inhibitory protein. These incorporated genes will direct transcription from a specific promoter that controls expression of the desired gene of interest. Additionally, genes can be under the control of a strong constitutive promoter.

It is noted herein that one of skill in the art will be able to select a promoter and expression control elements to selectively initiate transcription from pre-selected promoters, e.g., expression is initiated at promoters selected by one of skill in the art.

Current vectors utilize, for example, a temperature sensitive mutation in the CI protein, called CI₈₅₇, that allows the phage to grow as a lysogen at low temperatures and for CI₈₅₇ to become inactive and thereby induce lytic growth at higher temperatures (Hoffmann, F. et al., 1999, FEMS Microbiol. Lett., 177:327-34; Dodd, I. et al., 2001, Genes Dev., 15:3013-22). When the switch to a lytic pathway is initiated, the phage excise from the host genome, replicate its DNA, and eventually turn on the late transcript that is associated with host cell lysis and packaging of the phage genome into new viral particles. Such vectors merely delay host cell lysis without having the ability to precisely control host cell lysis while the phage is growing lytically. Again, a significant advantage of the vectors described herein is that it allows for precise control of host cell lysis after lytic growth has been established in the host cell.

Since the CI protein determines the switch between lysogenic or lytic development, other early phage genes necessary to rearrange macromolecular synthesis are shut off during lysogenic growth. The vectors described herein allow for lytic growth after infection, with an inducible promoter driving the expression of individual phage genes responsible for host cell lysis (“late” genes). Thus, the “early” phage-encoded proteins are active and able to alter host cell activity so that high yield of desired proteins can be achieved prior to host cell lysis or expression of “late” phage genes. The first example of this type of system is the production of protein in a phage strain containing an amber mutation in the S gene of phage λ (Panasenko, S. et al., 1977, Science, 196:188-9). The S gene encodes the holin protein so that the phage is allowed to develop lytically, from replication to generation of new viral particles, but lacks the ability to lyse the host cell. This mutation allows high levels of both protein of interest and phage particles to accumulate inside the cell. Systems based on amber mutations in the Q and S proteins also resulted in moderate expression levels of the heterologous protein (Murray, N. and Kelley W., 1979, Mol. Gen. Genom., 175:77-87; Lin, C. et al., 1998, Biotech. Bioeng., 57:529-35). This is due to the fact that the Q protein is responsible for control of the late transcript that carries all of the packaging and lysis proteins needed to create new viral particles and lyse the host cell (Roberts, J., 1975, Proc. Natl. Acad. Sci. USA, 79:3300-3304). However, such vectors only delayed lysis and do not allow for control of lysis, as host cell lysis follows soon after lytic growth is induced.

Vectors altering the λ S and E genes have been described (Murray, K., 1987, U.S. Pat. No. 4,710,463). An E mutation renders the phage deficient for packaging the phage genome into the viral capsid and thus allows for phage-encoded genes to be available for transcription for longer periods of time. However these S deficient expression systems also do not allow for the precise control of host cell lysis and release of the protein of interest, a component that helps to improve yield during the purification stages of protein production.

The intact S protein in previous X vectors eventual lysis of the host cell that is slowed by an amber mutations to the N, Q or R genes. What makes the S positive, N, Q, or R negative system devised by Kordyum, V. et al. (U.S. Pat. No. 6,268,178, U.S. Pat. No. 6,773,899, U.S. Pat. No. 6,794,162) unique, for example, is the use of a slowly lysing phage such that high expression yield is achieved in combination with the eventual lysis of the host cell and release of heterologous protein into the culture. The lysis delay was further enhanced by high multiplicities of infection and low culture temperatures to ensure activity of a temperature sensitive CI gene. Gene expression is also directed from both plasmid and phage vectors to enhance yield. In the most recent version, Kordyum et al. proposed to direct and control gene expression from a strong or inducible promoter such as the T7 promoters of the pET vectors or the lacUV5 promoter. Even this system, however, is not optimized for yield inasmuch as lysis itself is only delayed and not controlled by the user at a particular point in the lytic life cycle of the phage.

Additionally, despite the advantages related to high expression and release of soluble exogenous protein, none of the previous expression systems completely solve the issues related to leaky promoters. Most of these systems also require the use of plasmids to enhance protein yield in conjunction with inducible promoters for toxic gene expression. They also rely on temperature control of the CI protein for lytic activity of the vector and shutdown of many of the host cell processes resulting from a lytic bacteriophage, which leads to macromolecular shutdown of the host cell and eventual lysis of the host cell.

The recombinant phage vectors described herein will proceed through lytic development up to the point of activation of the late transcripts. In place of the late transcripts, the viral vector can the, for example, encode for control proteins that will shut off host expression and turn on expression of a desired gene from a specific foreign promoter. Late transcripts containing the viral capsid, packaging, and lysis genes are directed from an inducible promoter that can be induced by the user after optimal expression of the heterologous protein of interest. Late transcript repression is managed by the addition of repressible operators to the specific promoter controlling the late genes, e.g., the genes responsible for host cell lysis and phage packaging.

Once maximum expression of the desired protein is achieved, induction of the late transcripts can occur, for example, through the addition of a chemical inducer to the bacterial culture or by temperature induction. This induction will unlock the repressed late transcripts allowing for the production of new viral vectors as well as lysis protein that will release the desired protein product from the host cells.

In one embodiment, the phage will be based on the temperate λ bacteriophage. As the genome of bacteriophage λ is known, one of skill in the art can define the capsid (A-J), packaging (E), and lysis (S, R) proteins that make up the late transcript. In this preferred embodiment the switch from early to late transcripts is defined by the production of the Q protein. The late transcript directed by the Q protein will then produce proteins that will control subsequent RNA transcription. The control proteins that turn on desired gene transcription and shut off host transcription can be made up of, for example, phage-encoded sigma factors. An example of sigma factors that can be used are the T4 bacteriophage middle promoter control proteins, AsiA and MotA (Ouhammouch, M. et al., 1995, Proc. Natl. Acad. Sci. USA, 92:1451-5). Such factors will allow for host or foreign RNA polymerase to be directed solely to the expression of the heterologous protein of interest. In situations where a foreign RNA polymerase is utilized, the gene encoding the RNA polymerase can be, for example, expressed on aplasmid vector or inserted into the host cell genome by cloning or, for example, the use of a lysogenic vector. The control proteins can also consist of phage-derived RNA polymerases and an associated host RNA polymerase inhibitor. Alternatively, the use of proteins that phosphorylate the host RNA polymerase can also be used to help shut down host transcription. Such methods for optimizing expression of vector proteins are known in the art and can be used with the vectors described herein.

An alternative form of this vector places the late transcripts under control of repressible operators only, without the use of RNA polymerase control proteins or foreign RNA polymerases. Without the use of RNA polymerase control to drive expression, desired gene transcription are directed from strong constitutive promoters, for example, the AT-rich early promoters of the T4 and T5 phage.

The lytic pathway of the phage vectors described herein can be maintained by mutations that prematurely stop translation of the CI protein. These mutations can be true stop codons, amber mutations, or temperature sensitive mutations that affect the gene product activity, such as, for example, CI857. Additionally, the lytic pathway can be maintained by similar mutations to either the CII or CIII proteins.

For expression of a heterologous gene, at least one copy of the gene encoding for the desired protein of interest will be placed within the viral vector, for example, at a multiple cloning site (MCS) region of the vector, where it will be replicated to a high copy number during the lytic cycle of the phage or prior to phage vector infection. To further increase expression, plasmids having at least one copy of the desired gene can be maintained in the host cell prior to phage vector infection to help boost expression levels.

One of skill in the art will appreciate that any phage can be used to create a vector according to the present invention. For example, the vectors described herein can be based on all temperate and lytic bacteriophage of the siphoviridae, podoviridae, myoviridae, leviviridae, inoviridae, and microviridae families as well as any unclassified caudovirales that are capable of being rearranged to conditionally express the late transcripts necessary for production of structural phage components and host lysis proteins. Specifically, a phage-based expression vector can be designed with phage exhibiting a wide host range, e.g., coliphage, bacillus phage, pseudomonas phage, lactococcus phage or raistonia phage. Methods are known in the art for altering, for example, a phage genome such that genes responsible for host cell lysis and/or phage packaging can be placed under the control of an inducible promoter.

One of skill in the art will also know how-to determine the appropriate host cell range, e.g., the bacterial strains capable of being infected by the phage-derived vector, and therefore, one of skill in the art will be able to select an appropriate host cell. Specifically, a phage-based expression vector can be designed with phage based on coliphage, bacillus phage, pseudomonas phage, lactococcus phage or ralstonia phage. In these cases, the host cell must consist of the associated species of microbe. siphoviridae, podoviridae, leviviridae, and myoviridae families as well as any unclassified caudovirales. Vectors can be based on DNA or RNA bacteriophage.

Methods for expression or overexpression of exogenous or endogenous proteins are known to those of skill in the art, and these methods can be used in conjunction with the vectors described herein. Beginning with expression control it is noted that a basic form of expression control occurs at the transcriptional level. Transcriptional control is generally conducted through the use of strong promoters as well as inducible or repressible control elements. For example, protein expression from plasmids can be mediated by strong, regulated promoters that are native to the host organism such as the wild-type Lac or LacUV5 promoter. Even stronger promoters are known that contain consensus promoter sequences located at the -35 and -10 regions of transcription, such as the pTrc and pTac promoters (Lisser, S. and Margalit, H., 1994 Eur. J. Biochem., 223:823-30; Amann, E. et al., 1983, Gene, 25:167-178; Brosius, J. et al., 1985, J. Biol. Chem., 260:3539-41). These promoters were improved by using bacteriophage-derived promoter sequences that can out-compete these promoters due to AT-rich regions upstream of the -35 region. Examples of these AT-rich promoter are found in the T4 and T5 bacteriophage early promoter consensus sequences. The T5 promoter has been used commercially in plasmid vectors (Liebig, H., and Rüger, W., 1989, J. Mol. Biol., 208:517-536; Gentz, R. and Bujard, H., 1985, J. Bacteriol., 164:70-7; Bujard et al., 1985, U.S. Pat. No. 4,495,280; Banks et al., 1987, U.S. Pat. No. 4,689,406).

To further increase expression from strong promoters, promoter sequences can be used that are directed by foreign RNA polymerases or host RNA polymerases under the control of accessory proteins. The pET vector system is one example of the foreign polymerase control. It places the gene of interest under the phage-derived T7 promoter sequences, which are recognized by the phage T7 polymerase. Control of the host RNA polymerase by accessory proteins occurs through the action of, for example sigma and anti-sigma factors involved in promoter sequence recognition. In host cells such as, for example, E. coli, the consensus regions located at -35 and -10 are under control of the sigma70 subunit of the RNA polymerase complex. However, bacteria growing under conditions such as heat shock or bacteriophage infection can express alternative sigma factors or anti-sigma factors that interact with the host RNA polymerase or sigma 70 subunit. Instead of recognizing the -35 and -10 regions normally associated with the sigma70 control protein, these alternative control proteins direct the RNA polymerase to different promoter sequences (Missialcas, D. et al., 1998, Mol. Microbiol., 8):1059-66). The T4 bacteriophage middle promoter and its associated AsiA and MotA proteins fall into this sigma factor class, as these factors direct transcription to the T4 promoter. Specifically, the AsiA protein interacts with the sigma70 factor while MotA directs AsiA and the associated host RNA polymerase to an alternative promoter sequence near the -35 region. One advantage of a sigma factor-based transcriptional control system is that it utilizes the existing host RNA polymerase to turn on transcription from a designated promoter at the same time it shuts off host transcription from normal promoters. The ability to repress host transcription while enhancing specific promoter transcription and gene expression can be a very powerful tool in generating high levels of protein expression.

One of skill in the art will recognize the fact that strong promoters are often used in conjunction with transcriptional control sequences that allow for inducible or repressible regulation of expression. This is accomplished by placing inducible and repressible sequence elements upstream and downstream of the promoters and transcription start points. These elements allow regulation of transcription and expression to be managed by various chemical signals. The Lac operator element of the Lac and LacUV5 promoters is one known example of such an inducible or repressible control element. LacO operator sequences are recognized by a the LacI polypeptide that bind near the promoter sequences and physically prevent the RNA polymerase from initiating transcription (Muller, J. et al., 1996, J. Mol. Biol., 257:21-9; Muller-Hill, B., 1998, Curr. Opin. Microbiol., 1:145-51; Dubendorff, J. and Studier, F., 1991, J. Mol. Biol., 219:45-59). Induction of expression is accomplished by addition of lactose or lactose-like inducer such as IPTG (isopropylthiogalactoside), which binds to the LacI repressor protein and changes its conformation so that it cannot interact with and bind to the operator sequence. The operators that bind the tetr protein of the tetracycline controlled promoter act in a similar manner (Saenger, W. et al., 2000, Angew. Chem. Int. Ed. Engl., 39:2042-2052). Operators that bind araC protein are also controlled in this manner. Such chemically-inducible promoters can be used, for example, to control expression of the phage-derived vector late genes and/or expression of the protein of interest.

The use of single repressor sites and dual repressor sites an be used for the vectors and methods described herein. For example, dual lacI repressor sites around a sigma factor directed promoter, a foreign polymerase directed promoter or an AT-rich promoter can be used.

While it is important to note the repressible elements, there is another method for controlling host RNA polymerase. In various systems such as the pBAD promoter or Lac promoter, one finds recruitment of the host RNA polymerase by various upstream and downstream elements (Guzman, L. et al., 1992, J. Bacteriol., 177:4121-4130; Lai et al., 1991, U.S. Pat. No. 5,028,530). The particular feature of these systems is the CAP binding site, which is regulated by the catabolite activator protein. In the presence of cyclic AMP, dimers of the CAP protein interact with the alpha subunit of the RNA polymerase to help initiate transcription.

Induction systems based on recruitment of RNA polymerase can be repressed when an ADP-ribosyl-transferase modifies RNA polymerase alpha subunits. This mechanism phosphorylates the host RNA polymerase subunits such that upstream and downstream control elements and their associated activation proteins lose their ability to interact with the RNA polymerase and induce transcription. An example of this mechanism is seen in the T4 bacteriophage and its Alt, ModA and ModB proteins (Goldfarb, A. and Palm, P., 1981, Nucleic Acids Res., 9:4863-78). Repression by phosphorylation has yet to find its way into a commercial system such as a viral or plasmid vector but could be used to help suppress host transcription while allowing specific promoters to be activated by the control systems previously mentioned.

### Exemplification

### Example 1

This particular vector defines a protein expression system that is unique in several aspects. First, the vector is a viral vector based on a bacteriophage that has been rearranged to control the expression of the late phage transcripts in an “on/off” manner. Second, the vector contains an expression mechanism that utilizes RNA polymerase control proteins to redirect transcription. Third, the vector encompasses repressible elements that lock and unlock late phage transcripts based on chemical control.

Specific Design of Phage Vector

The platform of this vector utilizes a recombinant form of the temperate bacteriophage λ. Phage λ has been rearranged to remove portions of the genome that are essential for lysogenic development. Specifically, much of the region between approximately 22,000 and 32,000 has been removed (according to the reference sequence of wild-type bacteriophage λ, accession number NC_{—}001416).

The regions of λ that result in lytic development are left intact because of their ability to rearrange the macromolecular machinery of the host as the phage proceeds to replicate itself. Phage λ allows for such a rearrangement of the genome, and this can be accomplished with minimal affect to the lytic phage life cycle. In the case of this system, alteration of the CI gene using nonsense mutations ensures the lytic development of what was formerly a temperate phage. It should also be noted that bacteriophage λ infection induces the host cell to enter a quiescent-like state where the cell cycle is halted prior to cell division and the cell enlarges (Segueev, K. et al., 2002, J. Mol. Biol., 324:297-307).

Some of the “extra space” in the recombinant genome is occupied by an “expression cassette” that contains a site to insert a gene of interest, while other genomic space is occupied by a “control cassette” that contains proteins that control the host RNA polymerase. In the existing invention the placement of the expression cassette is in the removed region of the genome while the placement of the control cassette is inserted downstream of the λ late promoter, p′R. This ensures that the control cassette is not expressed until the cascade of lytic events leads to the production of the λ Q protein. Its is the Q protein that unlocks the late promoter, p′R, allowing for expression of the control proteins.

The placement and nature of the control cassette and the lack of a functional CI protein allows the phage vector to develop in this lytic manner up to the point of late transcription. This includes replication of the viral genome and progression from early to late gene expression as mediated by the λ N protein. The genes of the control cassette, however, are made instead of those of the late transcript that encodes phage capsid (A-J), packaging (E), and lysis (S, R) genes. The proteins of the control cassette direct the host RNA polymerase towards a promoter of choice and away from the host and phage promoters—in effect shutting down all non-essential transcription and associated translation.

The late transcript is intact, but under control of a conditional promoter. The conditional promoter is similar to the promoter of choice with the addition of repressor elements. These repressor elements ensure that the late transcripts are shut off until a desired time to be determined by the skilled user. The late transcript under this repressible promoter is designed to be “unlocked” (induced), in this example, by a chemical signal.

Unlocking of the late transcript results in production of new phage progeny and lysis of the host cell. The host cell lysis releases the gene product of interest (used interchangeably herein as “protein of interest” or “polypeptide of interest”) along with the new λ phage into the culture medium.

When late transcripts are induced, for example, by the addition of a suitable inducer, viral particles are made and host cell lysis occurs in a similar fashion to that of a fully competent phage. This releases new phage vectors as well as the protein of interest into the culture. Infection and lytic development of this recombinant phage leads to high levels of expression of soluble and biologically active heterologous protein that can be released from the host cell by chemical induction of the late viral transcripts.

The expression and control cassettes are made of specific promoter and gene components. Notably these two cassettes work together to “hijack” the host cell beyond the normal lytic processes of bacteriophage λ. The proteins found on the control cassette are the AsiA and MotA sigma factors of the T4 bacteriophage (reference sequences NP_{—}049866 and NP_{—}049873 respectively). These two proteins act to redirect RNA polymerase towards the T4 bacteriophage middle promoter. Therefore gene expression is controlled by placing desired genes and transcripts under direction of T4 middle promoters. While it is understood that in much of the literature, the sigma factors presented herein also fall under a anti-sigma factor classification, the term “sigma factors” is used herein to refer to all sigma and anti-sigma factors that interact with the RNA polymerase to direct transcription from a specific promoter.

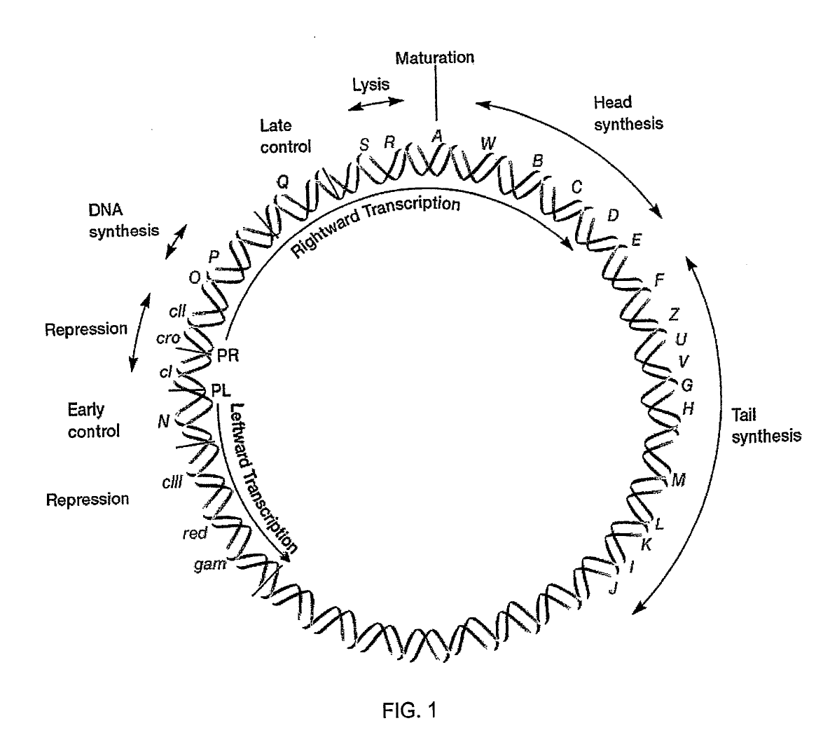

There are two T4 middle promoters located in this vector (see FIG. 1). First, the expression cassette contains a T4 middle promoter that directs expression of the gene of interest. Second, the control cassette contains a T4 middle promoter that is used to direct the late lambda transcript and its associated packaging and lysis genes (S through J). However, the late transcript T4 middle promoter is not transcribed once the control cassette proteins are expressed because it contains repressor elements. In the present example, lacI repressor sites of the lac promoter are used to control late gene expression. This requires the use of lactose or another chemical signal, such as IPTG, to act upon the lacI repressor and unlock the promoter. Dual lacI repressor sites are used to more tightly control the T4 promoter associated with the λ late transcripts. This ensures a strong repression of the promoter without the need for special host cells that contain an extra copy of the lacI repressor under constitutive expression (e.g., lacIq host strains of E coli). Expression of the protein of interest proceeds, therefore, until a chemical inducer is added to unlock the late transcript, thereby inducing cell lysis.

One of skill in the art will be able to determine specific conditions related to the host strain, host culture density, and multiplicity of infection. Looking specifically at host strain choice, this present vector can be used, for example, with any strain of E. coli capable of being infected by bacteriophage λ. This eliminates most strains of E. coli that have a lysogen inserted in its genome since most lysogens will inhibit infection during subsequent phage exposure.

For maximum protein expression, the infection takes place under high cell density conditions, because once infection occurs, the host cells will enter a quiescent state initiated by early phage gene expression, where the cell will expand in size but not proceed to cell division. The multiplicity of infection (moi) for viable virus needed to infect the cells and ensure that every cell has a copy of the λ vector is between about 1 and 100, preferably between about 2 and 20. The preferred embodiment of the phage vector has retained the λ super infection exclusion protein gene, which should prevent multiple infection of the same host cell and help keep the moi towards the lower side of that scale.

Expression continues until a desired level and quality of soluble protein is produced. Once that level is achieved, inducer is added to the culture to allow for late phage transcript to be made and translated. Induction of the late transcript results in production of new phage progeny and lysis of the host cell. The host cell lysis releases the protein of interest along with the new phage particles, e.g., packaged phage capable of infection, into the culture.

The process for propagating the recombinant phage vector containing the gene of interest requires a method with which to propagate the phage particles from a very low concentration to a concentration that will be effective enough to infect the high density expression culture at the required moi. Specific media conditions for both plating and culture growth are accomplished by the presence of a low dose of the chemical inducer, IPTG or lactose. Methods for producing such phage are known in the art.

The lacI-based repressor “leakiness” (minimal expression even in the absence of the chemical inducer) allows the system to utilize chemical induction in a variably dosed manner where the late transcript can be partially induced so the late transcript acts in a delayed or slow manner even when the sigma factor control mechanism has been activated. Phage grown in a culture containing a low dose of inducer can act similarly to that of a wild-type phage proceeding normally through the lytic development cascade. The right dosage, as determined by one of skill in the art, of inducer allows for the right timing of lytic events related to the late transcript and provides for a burst size that can maintain propagation of a phage infection.

### Example 2

One of skill in the art will appreciate how the system and its components can be rearranged, substituted, or added to in ways that would result in a workable system. The following are examples of such alterations.

Regarding the arrangement of the bacteriophage λ genome, the removed region of phage λ can be as large as the region from the J protein to the N protein (base references 20,000 to 35,000 on a sequence map of wild type λ). The expression cassette can be located anywhere in the phage genome as long as it does not interfere with transcription and translation of any of the proteins associated with lytic development of the phage. Also, the λ genome can be altered with respect to the CII and CIII proteins to render the phage lytic.

Temperate phage s will be easier to design because of the ability to remove the genes associated with lysogenic development. For example, the lytic phage T7 or T4 phage contain many late promoters that would need to be placed under inducible control. For other phage-based vectors, space is limited such that the genome needs to be altered to remove some or all of the late transcripts necessary for phage structural proteins, packaging proteins and lysis proteins. The removed transcripts can be placed on a bacterial artificial chromosome or plasmid in an associated host cell. This type of genomic rearrangement is also possible but not necessary when using the temperate and λ-like phage.

An additional variation regarding the genomic arrangement places the expression cassette or a supplemental copy of the expression cassette containing the gene of interest on a plasmid or bacterial artificial chromosome. This phage-plasmid cooperative design is similar to the CE6 phage system used for toxic gene expression or the Kordyum phage based expression systems (Miao, F. et al., 1993, Biotechnol. Prog., 2:153-9). However, the presented invention is different in that the phage acts to hijack host cell function and control host RNA polymerase using the aforementioned control cassette. Also, this invention allows for chemically controlled expression of the late transcript that can lyse the host at a desired point in time.

Variations in the expression and control cassettes are also envisioned, for example, with the type of control elicited by the control cassette. For example, this system could be designed to not have any specific control proteins, but utilize only repressor elements to prevent the late transcript from being made. Such a system could be designed to drive expression of the gene of interest or the late transcripts using only strong promoters that are naturally recognized by the host, such as the lacUV5 promoter or the AT-rich promoters of the, T4 and T5 phage. This system could omit the host RNA polymerase control mechanism, but would retain the late transcript repressor mechanism. Host hijack in this case can be enabled, for example, by using genes that encode proteins that phosphorylate the host RNA polymerase and shut off CAP induced promoters. Specifically, RNA polymerase phosphorylases such as, for example, Alt, ModA or ModB proteins of bacteriophage T4 can be used.

An alternate version of this vector also uses a foreign RNA polymerase and its associated host RNA polymerase inhibitors on the control cassette, such as those found on phage T7. Such a redesign requires placement of the gene of interest and the late transcripts under control of the associated promoter, which in this case is the T7 promoter. This disclosure is different from two perspectives. For example, the associated host RNA polymerase inhibitor encoded by the 0.7 gene and the 2.0 gene of phage T7 is used (LeClerc, J. and Richardson, C., 1979, Proc. Natl. Acad. Sci. USA, 10:4852-6).

Other inducible promoters are also provided. For example, the lacI repressor sites are replaced with sites that bind the araC protein or tetR protein of the arbinose and tetracycline induction/repression systems, respectively. Any chemically or temperature controlled repression system can be used to control late transcription.

While this invention has been particularly shown and described with references to preferred embodiments thereof, it will be understood by those skilled in the art that various changes in form and details may be made therein without departing from the scope of the invention encompassed by the appended claims.

What is claimed is:

1. A phage-derived vector for cloning or expressing a heterologous polypeptide of interest in a bacterial cell growing in culture, said vector comprising one or more factors that direct expression of a polypeptide of interest and at least one conditional promoter operably linked to coding sequences expressing one or more late phage transcripts, wherein expression of the one or more late phage transcripts cause host cell lysis, thereby releasing the expressed polypeptide of interest into the bacterial culture.

2. The vector of claim 1, wherein at least one conditional promoter is responsive to a chemical signal or a temperature signal, thereby inducing expression from the conditional promoter.

3. The vector of claim 2, further comprising one or more operator sequences that bind a repressor protein that is affected by a chemical signal.

4. The vector of claim 3, wherein the repressor protein is LacI.

5. The vector of claim 1, wherein the vector further directs expression of one or more factors that cause host RNA polymerase to selectively start transcription from pre-selected promoters.

6. The vector of claim 5, wherein one or more factors is selected from the group consisting of: AsiA, MotA, sigma factors, and anti-sigma factors.

7. The vector of claim 5, wherein the host RNA polymerase initiates transcription at a vector promoter.

8. The vector of claim 7, wherein the vector promoter is operably linked to a sequence encoding the polypeptide of interest.

9. The vector of claim 1, wherein the vector further comprises one or more promoters that are recognized by an exogenous RNA polymerase.

10. The vector of claim 9, wherein one or more promoters are the bacteriophage T7 promoter.

11. The vector of claim 1, wherein the sequence that encodes the polypeptide of interest is under the control of a promoter that is recognized by the exogenous RNA polymerase.

12. A phage particle comprising a phage-derived vector for cloning or expressing a heterologous polypeptide of interest in a bacterial cell growing in culture, said vector comprising one or more factors that direct expression of a polypeptide of interest and at least one conditional promoter operably linked to coding sequences expressing one or more late phage transcripts, wherein expression of the one or more late phage transcripts cause host cell lysis, thereby releasing the expressed polypeptide of interest into the bacterial culture.

13-22. (canceled)

23. A method for expressing a polypeptide of interest, comprising:
(a) infecting a host bacterial culture with a phage particle comprising a phage-derived vector, said vector comprising one or more factors that direct expression of a polypeptide of interest and at least one conditional promoter operably linked to coding sequences expressing one or more late phage transcripts, wherein expression of the one or more late phage transcripts cause host cell lysis;
(b) growing the infected bacteria under conditions such that a polypeptide of interest is expressed; and
(c) inducing lysis of the cells by inducing expression of the late phage genes, thereby releasing the expressed polypeptide of interest into the culture medium.

24. The method of claim 23, wherein induction in (c) is by a chemical signal or a change in temperature.

25-32. (canceled)

33. The method of claim 23, wherein one or more genes of interest are expressed in trans with respect to the vector.

34. The method of claim 33, wherein the one or more genes of interest are expressed from a source selected from the group consisting of: an endogenous gene of interest, a gene of interest inserted into the host genome from a lysogenic vector or through recombination, a gene of interest placed in a plasmid vector, and a gene of interest placed in a viral vector.

35. The method of claim 23, where the host cell comprises a gene that encodes an exogenous RNA polymerase.

36. The method of claim 35, wherein the RNA polymerase is from bacteriophage T7.

37. The method of claim 23, wherein one or more control elements are expressed in the host cell.

38. The method of claim 37, wherein expression of the one or more control elements is from a source selected from the group consisting of: an endogenous gene of interest, a gene of interest inserted into the host genome from a lysogenic vector or through recombination, a gene of interest placed in a plasmid vector, and a gene of interest placed in a viral vector.

39. The method of claim 37, wherein the one or more control elements is selected from the group consisting of: AsiA, MotA, sigma factors, anti-sigma factors, transcriptional repressors, altered transcriptional repressors, transcriptional activators and RNA polymerase phosphorylases.
